(19) United States
(12) Patent Application Publication
(10) Pub. No.: US 2010/0099910 A1
(43) Pub. Date: Apr. 22, 2010

(54) CARBOXYLIC ACIDS PREPARED USING A SALT-SPLITTING PROCESS

(75) Inventors: Xiangsheng S. Meng, Chanhassen, MN (US); Paraskevas Tsobanakis, Inver Grove Heights, MN (US)

Correspondence Address: CARGILL, INCORPORATED, P.O. Box 5624, MINNEAPOLIS, MN, 55440-5624, US

(73) Assignee: CARGILL INCORPORATED, Wayzata, MN (US)

(21) Appl. No.: 12/444,335

(22) PCT Filed: Oct. 3, 2007
(86) PCT No.: PCT/US2007/080300

§ 371 Date: Apr. 3, 2009

Related U.S. Application Data
(60) Provisional application No. 60/849,538, filed on Oct. 4, 2006.

(51) Int. Cl. C07C 55/22; C07C 53/124

(52) U.S. Cl. 562/590; 562/606

(57) ABSTRACT

Processes for preparing carboxylic acids in which a single phase mixture including an ammonium salt of a carboxylic acid is heated in the presence of a non-aqueous solvent to split the salt and form the acid. The acid may be dehydrated to form unsaturated counterparts.

### CROSS REFERENCE TO RELATED APPLICATION

This application claims the benefit of PCT Patent Application Ser. No. PCT/US2007/080300, filed Oct. 3, 2007, entitled CARBOXYLIC ACIDS PREPARED USING A SALT-SPLITTING PROCESS, which claims priority to U.S. Provisional Patent Application Ser. No. 60/849,538, filed Oct. 4, 2006, entitled CARBOXYLIC ACIDS PREPARED USING A SALT-SPLITTING PROCESS, which are hereby incorporated by reference in their entirety.

### FIELD OF THE INVENTION

This invention relates to preparing β-hydroxy carboxylic acids and derivatives thereof.

### BACKGROUND

Carboxylic acids, including hydroxy-functional carboxylic acids, are useful in a number of industrial processes, including the synthesis of polyesters and polyamides. Such acids may be produced from fermentation. In order to improve fermentation performance, a base such as calcium hydroxide is often added to the reactor to neutralize the acid. To recover the acid, the fermentation broth is acidified using a strong or weak acid, typically a mineral acid such as sulfuric acid. This process creates a by-product waste salt, such as calcium sulfate (gypsum). This approach can be costly for reasons including the costs of chemicals used, processing costs, the costs of removal and disposal of the waste salt, yield loss, and the introduction of additional impurities that may require separation from the carboxylic acid.

### SUMMARY

In general, a cost-effective process for producing carboxylic acids and derivatives, including hydroxy-functional carboxylic acids and derivatives, from readily available starting materials in high yields is described. It can be implemented as either a batch or a continuous process. Unlike processes that produce by-product salts that have to be removed and discarded, the described process produces ammonia or amines that can be recycled and re-reagents such as mineral acids or carbon dioxide, nor is it necessary to use elaborate distillation processes, including azeotropic distillations, to obtain the acid.

A process for preparing a beta-hydroxy carboxylic acid is described that includes combining an ammonium salt of a beta-hydroxy carboxylic acid, water, and a non-aqueous solvent other than an alcohol to form a single phase mixture, and heating the mixture to split the ammonium salt of the beta-hydroxy carboxylic acid and form a reaction product comprising the beta-hydroxy carboxylic acid and the non-aqueous solvent. The beta-hydroxy carboxylic acid can include 3-hydroxypropionic acid, 3-hydroxybutyric acid, or 3-hydroxyisobutyric acid. The beta-hydroxy carboxylic acid can include citric acid.

The non-aqueous solvent can have a boiling point greater than 100° C. when measured at a pressure of 1 atmosphere, or can have a boiling point greater than 175° C. when measured at a pressure of 1 atmosphere. The non-aqueous solvent can include a ketone, an amide, or an amine. The non-aqueous solvent can include sulfur. The non-aqueous solvent can be a solvent other than an alcohol.

The process can further include separating the beta-hydroxy carboxylic acid from the non-aqueous solvent. The process can further include dehydrating the beta-hydroxy carboxylic acid to form an unsaturated carboxylic acid (e.g., dehydrating 3-hydroxypropionic acid to form acrylic acid). The process can include heating the ammonium salt in the absence of an acidifying agent other than the acid of the ammonium salt to split the salt and form the reaction product.

A process for preparing a beta-hydroxy carboxylic acid is also described including combining an ammonium salt of a beta-hydroxy carboxylic acid, water, and a non-aqueous solvent other than an alcohol to form a single phase mixture, and heating the mixture to split the ammonium salt of the beta-hydroxy carboxylic acid and form a single phase reaction product comprising the beta-hydroxy carboxylic acid. The beta-hydroxy carboxylic acid can include 3-hydroxypropionic acid, 3-hydroxybutyric acid, or 3-hydroxyisobutyric acid. The beta-hydroxy carboxylic acid can include citric acid. The non-aqueous solvent can be a solvent other than an alcohol.

The process can further include separating the beta-hydroxy carboxylic acid from the non-aqueous solvent. The process can further include dehydrating the beta-hydroxy carboxylic acid to form an unsaturated carboxylic acid (e.g., dehydrating 3-hydroxypropionic acid to form acrylic acid). The process can include heating the ammonium salt in the absence of an acidifying agent other than the acid of the ammonium salt to split the salt and form the reaction product.

These and other objects and advantages of the present invention will be apparent to those skilled in the art from the following detailed description and claims.

### DETAILED DESCRIPTION

Carboxylic acids, including hydroxy-functional carboxylic acids, and derivatives thereof, may be prepared using a one-phase salt splitting system. An aqueous solution of an ammonium salt of a carboxylic acid is combined together with a non-aqueous solvent to form a single phase mixture. Heat is then applied to the single phase mixture to split the salt and form a carboxylic acid.

A fermentation process may be used to generate the carboxylic acid initially and form the aqueous solution. However, processes other than fermentation can also be used to provide the starting material. In addition, carboxylic acids other than hydroxy-functional carboxylic acids can be produced. Examples include mono-carboxylic acids (i.e., carboxylic acids having a single acid group such as acetic acid, propionic acid, butyric acid, and the like) and poly-carboxylic acids (i.e., carboxylic acids having more than one acid group such as succinic acid, fumaric acid, itaconic acid, and the like).

A fermentation reaction, typically involving the fermentation of a sugar such as glucose in the presence of a microorganism, takes place in a fermentation reactor to produce a fermentation broth containing a hydroxy-functional carboxylic acid. Any microorganism can be used, including, for example, the microorganisms described in U.S. Patent Application Publication No. 2004/0076982, published Apr. 22, 2004, entitled “3-Hydroxypropionic Acid and Other Organic Compounds,” which is incorporated by reference in its entirety. A base material can be added to a fermentation broth including carboxylic acid in a fermentation reactor to neutralize the acid, thereby forming an ammonium salt of the acid. In one embodiment, the base material can be ammonia or an amine. Suitable amines include primary, secondary, and tertiary amines. Specific examples of amines that can be used include trimethylamine, triethylamine, dibutylamine, diamines, and the like. Preferably, the base material has a boiling point no greater than 175° C. to facilitate subsequent removal from the reaction mixture.

A hydroxy-functional carboxylic acid includes at least one hydroxyl group and at least one carboxylic acid group. One class of useful hydroxy-functional carboxylic acids includes alpha-hydroxy carboxylic acids. Representative examples of such acids include lactic acid, citric acid, malic acid, tartaric acid, and glycolic acid. A second class of useful hydroxy-functional carboxylic acids includes beta-hydroxy carboxylic acids such as 3-hydroxypropionic acid, 3-hydroxybutyric acid, 3-hydroxyvaleric acid, 3-hydroxyhexanoic acid, 3-hydroxyheptanoic acid, and 3-hydroxyoctanoic acid. A third class of useful hydroxy-functional carboxylic acids includes gamma-hydroxy carboxylic acids such as 4-hydroxybutyric acid, 4-hydroxyvaleric acid, and 4-hydroxyhexanoic acid. A fourth class of useful hydroxy-functional carboxylic acids includes epsilon-carboxylic acids such as 5-hydroxyvaleric acid and 5-hydroxyhexanoie acid. Another example of a useful hydroxy-functional carboxylic acid is citric acid. Lactic acid and 3-hydroxypropionic acid are particularly preferred.

One or more non-aqueous solvent(s) can be added to the aqueous solution including the salt of the carboxylic acid to form a one-phase system with the aqueous solution. If the aqueous solution used includes a fermentation broth, the non-aqueous solvent may be added to the fermentation broth either in the reactor or after it leaves the reactor. Suitable non-aqueous solvents include those that can form a single phase with an aqueous solution, and therefore may be miscible or partly miscible with an aqueous solution of carboxylic acid. In one embodiment, the non-aqueous solvent is a solvent other than an alcohol. In addition, the non-aqueous solvent preferably has a relatively high boiling point so that it does not boil off during the extractive salt splitting, but remains with the acid. The non-aqueous solvent(s) used preferably have boiling points greater than 100° C. (measured at a pressure of 1 atmosphere) and more preferably greater than 175° C. (measured at a pressure of 1 atmosphere).

Examples of useful classes of non-aqueous solvents include ketones, amides, amines, and other solvents. Examples of suitable ketones that may be used include 2-pyrrolidinone, methyl pyrrolidinone, n-methyl-2-pyrrolidinone, and cyclohexanone. Examples of suitable amides that may be used include dimethylformamide, diethylformamide, and dimethylacetamide. Examples of suitable amines that may be used include ethanolamine, diethanolamine, triethanolamine, morpholin, and n-methylmorpholine. Examples of other solvents that may be used include dimethylsulfoxide, sulfolane, dimethylsulfolane, and dimethyl-2-imidazolidinone.

Any of the above-described solvents may be used alone or in combination with any other. For example, it may be useful to combine an amine with a ketone. For example, the combination of solvents may include a combination of a solvent that is fully-miscible and a solvent that is partly-miscible with the aqueous mixture.

In one embodiment, no other acids are added during the process. All steps of the process can be conducted in the absence of an acidifying agent other than the acid of the ammonium salt. As heat is used to split the salt forming the acid, rather than forming the acid by using an acid in an acid or salt replacement/conversion reaction, no other acids may be necessary.

Heat is applied to the system to thermally decompose the salt of the carboxylic acid into a free acid and a basic compound. This is often referred to as salt splitting. The reaction temperature and time used for the salt splitting are selected based upon the particular carboxylic acid reactant. The temperature should be high enough to accomplish salt splitting efficiently, yet below the temperature at which the carboxylic acid, non-aqueous solvent, or both decompose or otherwise degrade. In general, suitable reaction temperatures range from about 20° C. to about 200° C., with temperatures in the range from about 40° C. to about 120° C. being preferred. The reaction may be carried out at atmospheric pressure or under reduced pressure. Reduced pressures enable lower reaction temperatures to be used.

During heating, the salt of the carboxylic acid splits into a free acid and base material. Thus, the salt-splitting reaction produces a basic compound as a by-product, and the base material can be removed from the system and recycled for reuse. For example, if the salt of the acid is ammonium salt, the basic compound will be ammonia. In other examples, the basic compound recovered can be an amine. The basic compound may be separated and removed from the reaction mixture, e.g., by heating, applying a vacuum, adding an inert gas such as nitrogen, or a combination thereof to strip the basic compound (such as ammonia or amine) from the remainder of the reaction mixture. If desired, the basic compound can be recycled and added to a fermentation reactor. In addition, water can distill off the single phase mixture during the heating and the salt splitting reaction. In some embodiments, the water can be recovered and re-used. In some embodiments, the water may be recovered together with the base material.

The decomposition of the salt into the free acid and the basic compound is a reversible reaction. Thus, the released acid and basic compound can recombine to form the salt of the acid. The addition of the non-aqueous solvent reduces or prevents the recombination of the free acid with the basic compound. The non-aqueous solvent can form a single phase with the aqueous solution of the salt of the acid released from the salt splitting. The acidity of the acid in the non-aqueous solvent can be reduced compared to that in the aqueous solution, and at the same time, the basicity of the released basic compound in the non-aqueous solvent can also be reduced. Each reduction of the activities of the acid and basic compound in the non-aqueous solvent can reduce the recombination of the free acid and the basic compound and drive the salt splitting to completion. Furthermore, as the non-aqueous solvent can form a single phase with the released acid, the acid concentration (or loading) in the non-aqueous solvent can be very high. For example, using 2-pyrrolidinone as a non-aqueous solvent for the salt splitting of ammonium 3-hydroxypropionate, the 3-HP loading in 2-pyrrolidinone can exceed 40%.

The acid in the non-aqueous solvent can be recovered by selective extraction of the non-aqueous solvent using an organic solvent, or by selective extraction of the carboxylic acid in the non-aqueous solvent using an organic solvent. Alternatively, the acid and the loaded non-aqueous solvent may he used directly as an end product, or as a feedstock for additional processing.

In the case of hydroxy-functional carboxylic acids, the acids may be dehydrated, forming their unsaturated counterparts. In some embodiments, the hydroxyl group can be removed during dehydration. For example, 3-hydroxypropionic acid can be dehydrated to form acrylic acid. Similarly, 3-hydroxyisobutyric acid can be dehydrated to form methacrylic acid.

Dehydration may be effected by heating the mixture containing the acid. A catalyst may be added to facilitate the dehydration process. Examples of suitable catalysts include acids (e.g., mineral acids, resins), bases (e.g., amines, metal hydroxides, resins), and neutral catalysts such as calcium phosphate salts, calcium lactate salts, metal oxides such as aluminum oxide, titanium oxide and zirconium dioxide, silicon dioxide, copper chromites, zeolites, and the like. The catalysts can be added and mixed with the mixture containing the acid, or the mixture containing the acid can be passed into or through a bed or column that contains the catalyst. The dehydrated product may be purified by distillation.

In one embodiment, a 3-HP loaded non-aqueous solvent is fed into a reactive column packed with a dehydration catalyst. Under the dehydration conditions (e.g. 180°-200° C.), the formed acrylic acid from the dehydration of 3-HP is vaporized and distilled out of the top of the column. The non-aqueous solvent passes through the column, and is collected off the bottom of the column. The collected non-aqueous solvent may be recycled for reuse. For example, a 3-HP loaded non-aqueous solvent may be used directly in the production of acrylic acid by dehydration of 3-HP. The formed acrylic acid has a much lower boiling point than 3-HP and the non-aqueous solvent, and so can be recovered simultaneously with the dehydration process.

The invention will now be described further by way of the following examples. The invention will be more readily understood by reference to the following examples. There are, of course, many other forms of this invention which will become obvious to one skilled in the art, once the invention has been fully disclosed, and it will accordingly be recognized that these examples arc given for the purpose of illustration only, and are not to be construed as limiting the scope of this invention in any way.

### EXAMPLES

### Example 1

60.0 g of a 29.8% ammonium 3-hydroxypropionate aqueous solution and 20.0 g of n-pyrrolidone were charged to a 500 ml three-necked flask. The flask was prepared with a stirring bar, heating mantle, thermocouple, and connected to a vacuum pump. The two solutions in the flask formed a homogeneous phase.

The flask was heated while the solution in the flask was stirred. When the temperature reached 80° C., the vacuum pump was turned on to produce a reduced pressure of 200 torr. Water in the solution started to evaporate and was removed under reduced pressure to a cool temperature trap. At the same time, ammonia from the decomposition of ammonium 3-hydroxypropionate was also released and removed under reduced pressure to a cool temperature trap. it required about 20 min. for most of the water to evaporate from the flask under about 200 torr reduced pressure. The flask was then heated to 120° C. at a reduced pressure of 75 torr for 10 min. The flask was then maintained at the same temperature under 10 torr for another 10 min. After this time, the flask was cooled to room temperature.

The remaining solution in the flask was analyzed by titration for the resulting concentrations of free 3-hydroxypropionic acid and ammonium 3-hydroxypropionate. The concentration of 3-hydroxypropionic acid in n-pyrrolidone was 41.26%. There was also 4.11% 3-hydroxypropionic acid existing as ammonium 3-hydroxypropionate in n-pyrrolidone. Therefore, the conversion of ammonium 3-hydroxypropionate to 3-hydroxypropionic acid was 90.9%.

### Example 2

A 1000-m1 3 necked flask was prepared as in Example 1. 200.0 g of a 29.8% ammonium 3-hydroxypropionate aqueous solution and 100.0 g of methyl pyrrolidone were charged to the flask. The solutions formed a homogeneous phase.

The procedure described in Example 1 was followed, with the following changes. After heating at 80° C. at 200 torr for 20 minutes, the formed single-phase solution in the flask was heated to 120°C. at a reduced pressure of 35 torr for 15 min. After this time, the flask was cooled to room temperature.

The solution was then analyzed as in Example 1. The result included a solution having a concentration of 39.04% 3-hydroxypropionic acid in methyl pyrrolidone. The remainder was ammonium 3-hydroxypropionate in methyl pyrrolidone. The final conversion rate obtained was 93.8% from ammonium 3-hydroxypropionate to 3-hydroxypropionic acid.

This invention has been described above in detail with particular reference to preferred embodiments thereof, but it will be understood that variations and modifications other than as specifically described herein can be effected within the spirit and scope of the invention. Moreover, all patents, patent applications, provisional patent applications, and literature references cited above are incorporated hereby by reference for any disclosure pertinent to the practice of this invention.

What is claimed is:

1. A process for preparing a beta-hydroxy carboxylic acid, comprising:
combining an ammonium salt of a beta-hydroxy carboxylic acid, water, and a non-aqueous solvent to form a single phase mixture; and
heating the mixture to split the ammonium salt of the beta-hydroxy carboxylic acid and form a reaction product comprising the beta-hydroxy carboxylic acid and the non-aqueous solvent.

2. A process according to claim 1, wherein the beta-hydroxy carboxylic acid comprises 3-hydroxypropionic acid.

3. A process according to claim 1, wherein the beta-hydroxy carboxylic acid comprises 3-hydroxybutyric acid.

4. A process according to claim 1, wherein the beta-hydroxy carboxylic acid comprises 3-hydroxyisobutyric acid.

5. A process according to claim 1, wherein the beta-hydroxy carboxylic acid comprises citric acid.

6. A process according to claim 1, wherein the non-aqueous solvent has a boiling point greater than 100° C. when measured at a pressure of 1 atmosphere.

7. A process according to claim 1, wherein the non-aqueous solvent has a boiling point greater than 175° C. when measured at a pressure of 1 atmosphere.

8. A process according to claim 1, wherein the non-aqueous solvent comprises a ketone.

9. A process according to claim 1, wherein the non-aqueous solvent comprises an amide.

10. A process according to claim 1, wherein the non-aqueous solvent comprises an amine.

11. A process according to claim 1, wherein the non-aqueous solvent comprises sulfur.

12. A process according to claim 1, further comprising separating the beta-hydroxy carboxylic acid from the non-aqueous solvent.

13. A process according to claim 1, further comprising dehydrating the beta-hydroxy carboxylic acid to form an unsaturated carboxylic acid.

14. A process according to claim 2, further comprising dehydrating the 3-hydroxypropionic acid to form acrylic acid.

15. A process according to claim 1, wherein the ammonium salt is heated in the absence of an acidifying agent other than the acid of the ammonium salt to split the salt and form the reaction product.

16. A process according to claim 1, wherein the non-aqueous solvent is a solvent other than an alcohol.

17. A process for preparing a beta-hydroxy carboxylic acid, comprising:
combining an ammonium salt of a beta-hydroxy carboxylic acid, water, and a non-aqueous solvent other than an alcohol to form a single phase mixture; and
heating the mixture to split the ammonium salt of the beta-hydroxy carboxylic acid and form a single phase reaction product comprising the beta-hydroxy carboxylic acid.

18. A process according to claim 17, wherein the beta-hydroxy carboxylic acid comprises 3-hydroxypropionic acid.

19. A process according to claim 17, wherein the beta-hydroxy carboxylic acid comprises 3-hydroxybutyric acid.

20. A process according to claim 17, wherein the beta-hydroxy carboxylic acid comprises 3-hydroxyisobutyric acid.

21. A process according to claim 17, wherein the ammonium salt is heated in the absence of an acidifying agent other than the acid of the ammonium salt to split the salt and form the reaction product.

22. A process according to claim 17, wherein the non-aqueous solvent is a solvent other than an alcohol.
